(19) United States
(12) Patent Application Publication
(10) Pub. No.: US 2013/0190592 A1
(43) Pub. Date: Jul. 25, 2013

(54) METHODS AND SYSTEMS FOR DETERMINING THE VOLUME OF EPICARDIAL FAT FROM VOLUMETRIC IMAGES

(71) Applicant: CONSIGLIO NAZIONALE DELLE RICERCHE, Roma (IT)

(72) Inventors: Giuseppe Coppini, Calci (Pisa) (IT); Riccardo Favilla, Vecchiano (Pisa) (IT); Paolo Marraccini, Pisa (IT); Ovidio Salvetti, Ghezzano (IT); Davide Moroni, Calci (Pisa) (IT); Gabriele Pieri, Calci (Pisa) (IT)

(73) Assignee: CONSIGLIO NAZIONALE DELLE RICERCHE, Roma (IT)

(21) Appl. No.: 13/742,411

(22) Filed: Jan. 16, 2013

(30) Foreign Application Priority Data
Jan. 17, 2012 (IT) TO2012A000030

(51) Int. Cl. G06T 11/00; A61B 5/02

(52) U.S. Cl. CPC G06T 11/003; A61B 5/02
USPC 600/407; 382/131

(57) ABSTRACT

Methods and systems are provided for determining the volume of epicardial fat of a heart, which include acquiring a plurality of volumetric image data representing the heart; morphologically characterizing a volume of interest represented by the volumetric image data, including identifying a predetermined number of reference contours of the heart; estimating the epicardial surface, Σ, according to a three-dimensional series expansion of vector spherical harmonic functions based on the reference contours; and determining the volume of epicardial fat based on the voxels which are located inside the estimated epicardial surface, Σ, and which have a grey level within a predetermined range, characteristic of fatty tissue.

### CROSS REFERENCE TO RELATED APPLICATION

This application claims priority to and benefit of Italian Patent Application No. TO2012A000030 filed Jan. 17, 2012, the contents of which are incorporated by reference in its entirety.

### FIELD OF THE INVENTION

The present invention relates to quantitative clinical diagnostic methods and systems, particularly for image processing, and more particularly for the determination of real dimensional parameters of anatomical structures from volumetric images such as tomographic or magnetic resonance images, which can differentiate fatty tissues from other tissue components.

### BACKGROUND OF THE INVENTION

Pericardial fat, like other visceral fat, is correlated with important cardiovascular and metabolic pathologies and is considered to be a significant indicator of the risk factors. Numerous clinical studies have demonstrated the importance of the measurement of epicardial fat as an independent prognostic risk factor. The accumulation of visceral fat, and particularly epicardial fat, is considered to be a condition which promotes the inappropriate production of adipose endocrine factors such as adipokines which may affect tissue remodelling, the activation of inflammatory processes, and cardiac function. The quantification of epicardial fat can provide an independent objective parameter which is useful for prognostic stratification and longitudinal cardiovascular risk evaluation.

However, there are as yet no known methods for making a reliable and reproducible measurement of the volume of cardiac fat which are universally accepted and simple to use.

To this end, numerous studies have been conducted, using echocardiographic image acquisition, magnetic resonance imaging and tomographic imaging (cardiac CT).

Cardiac CT can be used to acquire images of the whole volume of the heart over the whole cardiac cycle, with good spatial resolution and excellent density resolution. The coronary vessels can thus be visualized by non-selective angiography procedures, and this has promoted the widespread use of cardiac CT methods. Numerous clinical studies have been conducted using computer tomography to evaluate the extent of fatty deposits. In these studies, the evaluation of fat was typically conducted by manually segmenting the two-dimensional images. However, this approach is subject to numerous sources of variability and is not suitable for routine use.

Other studies are limited to the simple evaluation of the thickness of the fat layer, which does not always provide an adequate and objective measurement of the volume of cardiac fat.

There are also known methods of estimating the volume of cardiac fat based on image processing methods. For example Bandekar, A. N., Naghavi, M., and Kakadiaris, I. A., in “Automated Pericardial Fat Quantification in CT Data,” Engineering in Medicine and Biology Society, 2006. EMBS '06. 28th Annual International Conference of the IEEE, pp. 932-935, (2006), describe an automatic method based on textural features, which provides an estimate of the total mediastinal fat. Similar results were obtained by Damini Dey, Yasuyuki Suzuki, Shoji Suzuki, Muneo Ohba, Piotr J. Slomka, Donna Polk, Leslee J. Shaw, and Daniel S. Berman, in “Automated Quantization of Pericardiac Fat From Noncontrast CT”, Investigative Radiology Volume 43, number 2, February 2008, using an approach based on multi-threshold region growing methods. Additionally, Coppini G, Favilla R, Lami E, Marraccini P, Moroni D, and Salvetti O., in “Regional epicardial fat measurement: Computational methods for cardiac CT imaging”, Transactions on Mass-Data Analysis of Images and Signals, Vol. 1:101-110 (2009), and Coppini G, Favilla R, Marraccini P., Moroni D., and Pieri G., in “Quantification of Epicardial Fat by Cardiac CT Imaging”, The Open Medical Informatics Journal, vol. 4 pp. 126-135 (2010), describe methods for manual segmentation of the epicardial surface, where the epicardial fat is then extracted automatically using a model based on level sets theory. It should be noted that the last two studies are based on manual slice-by-slice extraction of the contours of the heart, and the approach followed is strictly two-dimensional. In particular, the 2D images corresponding to each slice are processed individually in order to calculate the area corresponding to fat, while the total volume is calculated by integrating the values of the areas.

Unfortunately, at the present time there is no known method for measuring the volume of epicardial fat from tomographic scans or other volumetric images which can be used in clinical practice and is suitable for handling large numbers of cases.

### SUMMARY OF THE INVENTION

An object of the present invention is therefore to provide an easily used method for obtaining a reliable and reproducible measurement of the volume of cardiac fat, based on volumetric images, such as tomographic images, or on a plurality of available standard scans obtained by computer tomography of the heart, acquired with or without the use of contrast medium, for example in the course of coronary CT angio examinations, the method thus being usable in clinical practice.

According to the present invention, this object is achieved by methods for determining the volume of epicardial fat as described herein.

The present invention also includes systems for determining the volume of epicardial fat, a corresponding computer program, and a computer program product, as described herein.

Briefly, the present invention utilizes a three-dimensional model of series expansion of vector spherical harmonic functions from a series of volumetric images, such as tomographic scans comprising the cardiac volume, acquired with or without contrast medium for estimating the epicardial surface, and calculates the cardiac fat volume on the basis of the volume elements or voxels which are located within the epicardial surface estimated in this way and which have an attenuation level or grey levels within a predetermined range which is characteristic of fatty tissue.

Starting from the collection of images, the estimation of the epicardial surface may be guided by the preliminary identification, which may be manual or automatic, of a plurality of reference contours of the heart, representative of the heart as a whole, for example the contour of the heart which can be derived from a pair of views taken on a long axis (four chambers and two chambers), and from at least a pair of views taken on a short axis, extracted in the basal and apical regions.

The coefficients of the series expansion of vector spherical harmonic functions may be calculated by minimizing a predetermined cost functional.

Further characteristics and advantages of the invention will be disclosed more fully in the following detailed description with reference to the attached drawings.

### BRIEF DESCRIPTION OF THE FIGURES

FIG. 1 is a schematic representation of a processing system programmed for the implementation of a representative method according to the invention,

FIG. 2 is a flow diagram of a representative method according to the invention,

FIG. 3 shows a diagram of the heart with the positions of the various planes of interest and examples of images of the corresponding tomographic cross sections,

FIG. 4 shows a series of tomographic images representing differently orientated views of the heart, on which reference contours of the heart are shown,

FIG. 5 is a diagram representing the distribution of grey levels of the voxels within the estimated surface of the heart,

FIG. 6 shows two three-dimensional views (a left-hand front view and a righthand rear view) of epicardial fat reconstructed from tomographic images, processed by a representative method according to the invention;

FIG. 7 shows, from the top left to the top right, two-chamber and four-chamber views with corresponding lines of the atrioventricular plane, and, at the bottom, two views on a short axis with the lines of the interventricular plane.

FIG. 8 shows (i) a plate with four three-dimensional views of epicardial fat in the ventricular region, and (ii) views of the right and left cross sections of the ventricular fat deposits, according to a representative method of the invention.

### DETAILED DESCRIPTION

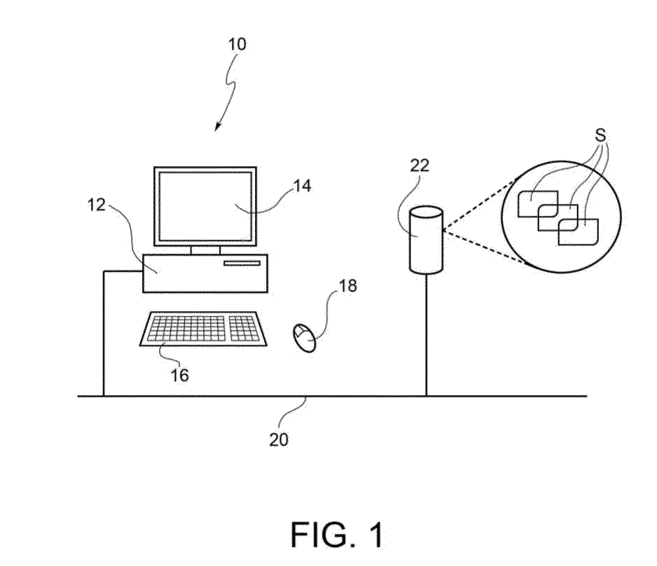

With reference to FIG. 1, a system for determining the volume of epicardial fat from volumetric images, including but not limited to tomographic images, is shown in its basic outlines. It may include, for example, a computer workstation 10 of a known type, having a processor subsystem (base module) 12, a display device 14, a keyboard 16, a pointing device (mouse) 18 and a device for connection to a local network (network bus) 20.

An example of a workstation 10 which can be used is the Mac Mini model, made by Apple, Inc., which has an Intel Core 2 Duo 2.0 GHz CPU, 4 GB RAM, a 250 GB hard disc and a MacOsX 10.6 operating system.

The workstation may be arranged to run a program or groups of programs stored on the hard disc or accessible via the network, and to show the results on the display 14. The program or groups of programs may be processing and calculation programs which implement the methods according to the invention, as will be described in detail below.

Systems according to the invention may further comprise a storage memory subsystem 22, of a known type, integrated with the workstation 10 or connected thereto by the network connection 20, and adapted to store databases of tomographic images, as described in detail below, with reference to the implementation of methods according to the invention.

Databases may be stored, if they are of limited size, in the hard disc of the workstation 10 without any modification of the characteristics of the invention. The systems also may be arranged for connection to other peripheral input/output devices, local or remote, or may include of other computer system configurations, such as a multiprocessor system or a computer system of the distributed type, where the tasks may be executed by remote computer devices interconnected by a communications network and where the modules of the program may be stored in both local and remote storage devices.

The invention further includes computer programs or group of programs, in particular computer programs on, or in, a data medium or memory, adapted to implement the invention. Such program may use any programming language, and may be in the form of source code, object code or a code intermediate between source and object code, for example, in a partially compiled form, or in any other desired form for implementing methods according to the invention.

Finally, embodiments of the invention further comprise computer program products, which may be in the form of computer storage medium readable by computer systems and which encodes a program or group of programs of computer instructions for executing the processing of data representing volumetric images. Specific non-limiting examples of a computer-readable data medium include any object or device capable of storing a program, such as random access memory, read-only memory, compact disc memory, or a magnetic recording medium or a hard disc. More generally, the computer program product may also be in the form of a data stream readable by a computer system, which encodes a computer instructions program, and which can be carried, for example, on a network, such as the Internet.

The systems described above are considered to be well-known in the art and thus will not be described further here.

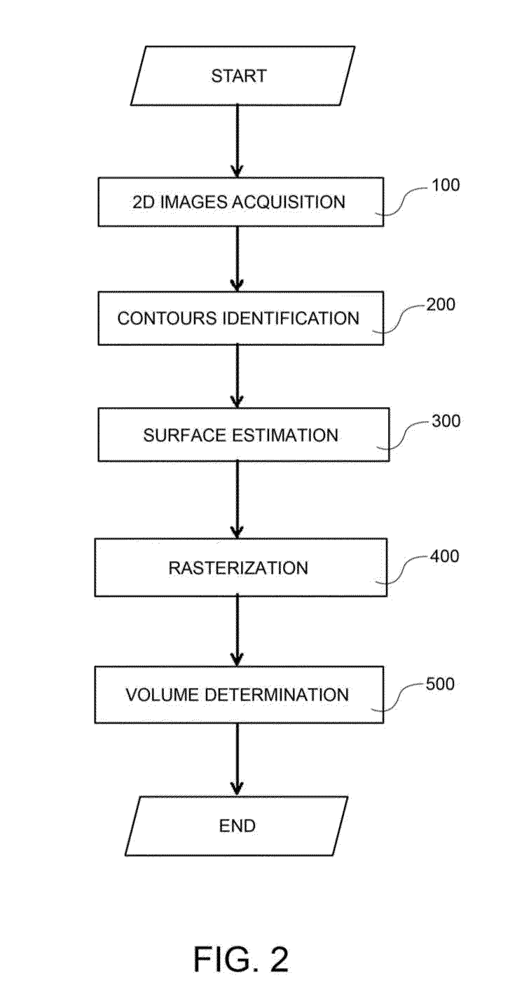

With reference to the flow diagram of FIG. 2, methods according to the invention may include initially, in step 100, the acquisition of a collection of two-dimensional image data, such as computer tomography image data S, representing a plurality of views, for example successive plane views (or slices) of the heart, typically also including images of adjacent organs or tissues. The collection of image data may be, for example, obtained at a time other than the time of implementation of such methods, and, as mentioned above, may be stored in the storage memory subsystem 22.

In general, a complete cardiac CT scan may be defined by a series of two-dimensional images acquired orthogonally to the axis of the scanning device (axis z). Each of these images represents attenuation of the scanning X-rays in a tissue slice of predetermined thickness (in the present case, typical thicknesses range from about 2.5 mm to about 0.5 mm). Considered as a whole, stacked along the axis z, the slices can define a function I(x,y,z) in the reference system of the scanning device in which I is the value of the attenuation, typically expressed in Hounsfield units (HU), at the point with coordinates x,y,z. The individual images (slices) generally correspond to I(x,y,z) for fixed values of z.

When tomographic images have been acquired, they may be initially converted into a suitable format for processing by an image representation and displaying program.

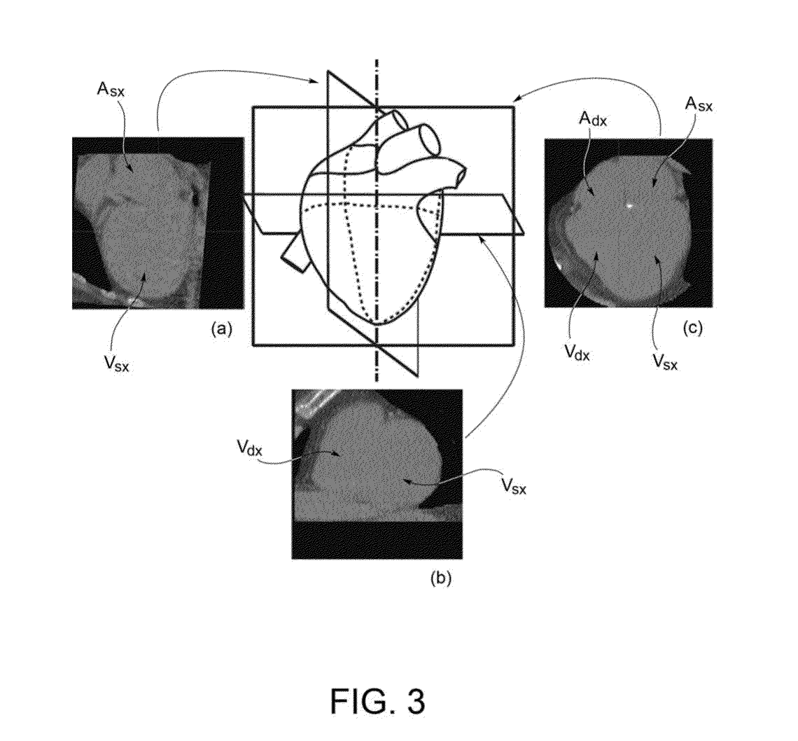

Starting with images S selected from the available series, including the whole cardiac volume, an operation (200) of morphological characterization of the volume of interest may be carried out, including the identification of at least three two-dimensional contours P1-P3 (and more generally a number n of profiles P1-PN) of the heart, this identification being performed, respectively, in at least one sectional image taken along a short axis at the base of the ventricle and in two sectional images taken along a long axis, (representing four chambers and two chambers respectively) of the heart. FIG. 3 shows diagram of a heart and along with cross-sectional planes of interest. In FIG. 3, the images (a), (b) and (c) show representative examples of CT images which are, respectively, (a) a sectional image taken on the long axis, showing the two left chambers, namely the left atrium Asx and the left ventricle Vsx, (b) a sectional image taken on the short axis, showing the two lower chambers, namely the right ventricle Vdx and the left ventricle Vsx, and (c) a sectional image taken on the long axis, showing all four chambers, namely the right atrium Adx, the left atrium Asx, the right ventricle Vdx, and the left ventricle Vsx.

Because of the regularity and symmetry (even if only approximate) of the epicardial surface, the use of other contours from long-axis images is typically not useful. With respect to the use of other contours from short-axis cross sections, it has been found experimentally that the addition of another contour in the medio-apical position may improve the estimate in individual cases. However, experimental observations suggest that there is typically no additional benefit in using more than three contours obtained in a short axis. It should also be borne in mind that an increase in the number of contours (and therefore of points used) generally leads to an increase in calculation time.

The number of contours to be traced therefore typically varies from a minimum of 3 (2 long-axis and 1 short-axis), sufficient for hearts with a regular anatomy, to a maximum of 5 (2 long-axis and 3 short-axis). Thus, the use of two long-axis sections and two short-axis sections appears to be sufficient for general use.

In view of the above considerations, in certain embodiments, the selected sections include at least one short-axis section taken in a basal region (optionally a short-axis section in the medio-apical region may be used), a long-axis section corresponding to the four-chamber view, and a long-axis section corresponding to the two-chamber view, corresponding to the left ventricle and atrium.

The contours may be identified automatically, for example by implementing an automatic shape recognition method such as a method for automatic segmentation of the cardiac volume, from the converted image data, and executed on the basis of algorithms described in the literature, or in assisted (semi-automatic) mode, or again by manual operation performed by an operator for plotting a contour curve marking a discrete number of boundary points. In assisted or automatic mode, the segmentation method may make use of the image gradient to locate the contours, subject to the limitations of current methods which may allow reliable recognition only for certain regions of the contour (such as the boundary between the left ventricle and the lung).

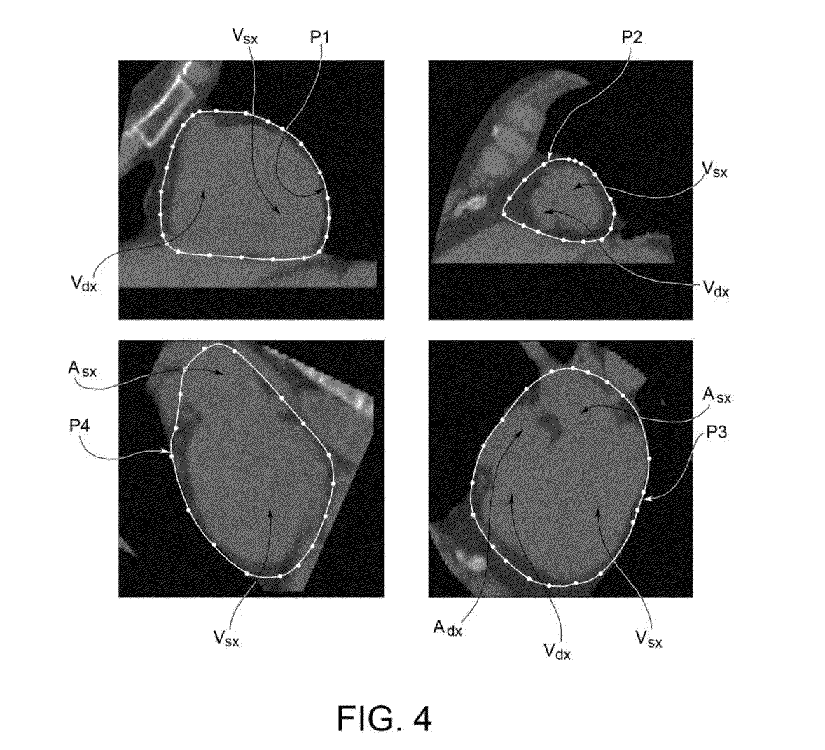

FIG. 4 shows (in clockwise order starting from the upper left) four images which include the volumes of interest, namely a first short-axis section of the basal region of the left ventricle Vsx and the right ventricle Vdx, a second short-axis section of the apical region of the same left ventricle Vsx and right ventricle Vdx, a first long-axis section including the view of all four chambers Asx, Adx, Vsx, Vdx, and a second long-axis section including the view of two chambers, namely the left atrium Asx and the left ventricle Vsx.

The contour curves P1-P4, which interpolate a predetermined number of discrete boundary points using cubic splines, are plotted on the images of FIG. 4. In certain embodiments, about 20 boundary points may be used to identify the long-axis contours, about 15 boundary points may be used for a short-axis section in a basal region, and about 10 boundary points may be used for a short-axis section in an apical region.

D={Di, i=1, . . . , K} denotes the set of all the points of the contours plotted in the common reference system.

In the next step 300, a 3D estimate may be made of the epicardial surface of the whole pericardium, from the points in D of the plotted contours. For this purpose, the centroid G of the points in D may be calculated, and the origin of the reference system may be translated to G. The spherical coordinates relative to the pole G also may be indicated by (φ,θ). In the following text, the symbol Σ denotes the epicardial surface and Ω denotes the domain enclosed by it. As a general rule, E may be expressed in parametric form as x(φ,θ), and may be represented here by a series of spherical harmonics.

$$x ( ϕ , θ ) = {\sum }_{n = 0}^{N} {\sum }_{m = 0}^{n} [ {a}_{mn} cos m ϕ + {b}_{mn} sin m ϕ ] {P}_{mn} ( cos ( θ ) )$$

where aₘₙ, bₘₙ are the (vector) coefficients of the series and Pₘₙ(t) are the associated Legendre polynomials of degree n and order m. The number N is the order of the surface. In the present case, in view of the (approximate) regularity and symmetry of the epicardium, a low order N should be suitable: experimental evidence indicates that a value of N of 2-3 is typically suitable for this purpose. However, for the procedure of estimating the coefficients of the series of spherical harmonics as described below, it is not necessary to specify the “exact” value of the order in advance. Nevertheless it should be borne in mind that implementation will be more efficient when the order of the surface is limited than when the order is too high, and this will also advantageously limit the calculation time. For this reason, N=3 is used in this embodiment.

The aim is find a regular surface which approximates the points in D. It is also assumed that the voxels within the surface can represent blood (or contrast medium if appropriate), cardiac muscle and epicardial fat: it is possible to identify with certainty a value I_{high} of the grey levels which is accepted as belonging to n. Similarly, it is possible to identify a value I_{low} below which the voxels cannot belong to n. Another piece of information which may be used relates to the regions where there are high grey level gradients (the heart-lung interface) which belong in almost all cases to the epicardial surface.

On the basis of these considerations, the coefficients of the series may be estimated by numerically minimizing the following cost functional:

C(aₘₙ,bₘₙ)=αP(aₘₙ,bₘₙ)+βG′(aₘₙ,bₘₙ)+γE(aₘₙ,bₘₙ)+δS(aₘₙ,bₘₙ)

in which the coefficients α, β, γ, δ may be selected as explained below, while the terms P, G′, E, S are defined as follows:
P is the approximation error, calculated as the sum of the distances from the surface Σ of the points Pᵢ, according to the following expression:

$$𝒫 = \frac{1}{K} {\sum }_{i} {d}_{i}^{2} ( {D}_{i} , Σ )$$

where K is the number of points Dᵢ
G′ penalizes the presence within the surface of “unexpected” grey levels, according to the expression

G′=∫_{Ω}H(I_{high}−I(x,y,z))(I_{high}−I(x,y,x))²dxdydz++∫_{Ω}H(I(x,y,z) . . . I_{low})(I_{low} . . . (x,y,z))²dxdydz

where H is the unitary step function and I_{low} and I_{high} represent, respectively, the lower and upper bounds of the grey levels of the fat window (for example, in the case of CT, I_{low}≈−200 HU and I_{high}≈40 HU). It should be noted that the first function to be integrated is cancelled for I(x,y,z)>I_{high}, while the second is cancelled for I(x,y,z)<I_{low}.
E indicates the coincidence of the surface with the regions having high grey gradients (heart/lung interface), according to the expression:

$$ℰ = {\int }_{Σ} \frac{1}{1 + {\vert ∇ I ( x , y , z ) \vert}^{2}} \vert ∇ I ( x , y , z ) \vert d Σ$$

the integral being calculated on the surface Σ. In the low-gradient regions it has a considerable weight, while at the edges the contribution to the integral is lower. It appears to be convenient to use the Gaussian gradient for the calculation of the gradient.
S ensures the regularity of the surface, by causing the coefficients of the series to decrease rapidly as the order increases, according to the expression:

$$𝒮 = {\sum }_{n} {\sum }_{m} {w}_{n m} ( {a}_{m n}^{2} + {b}_{mn}^{2} )$$

In particular, it is assumed that wₘₙ=k exp(n/N), which results in an exponential decrease in the coefficients of the series.

The coefficients α, β, γ, δ may be selected on the understanding that the constraints of approximation and regularity are stronger, since they represent advance knowledge of the problem, while the constraints relating to the image characteristics (grey levels and gradients) are weaker and subject to more uncertainty. Consequently, given that α, δ=1, the other two weights were selected as follows: β, γ=10⁻².

It should be noted that, generally speaking, the constrains of approximation and regularity P (aₘₙ, bₘₙ), S (aₘₙ, bₘₙ) are sufficient for estimating the epicardial surface Σ. This may be obtained by minimizing a more general cost function having the expression:

C(aₘₙ,bₘₙ)=P(aₘₙ,bₘₙ)+λS(aₘₙ,bₘₙ)

where the coefficient λ serves to control the degree of regularity and may be expressed as λ=1. In view of its greater computational simplicity, the use of the general cost function provides a decrease in the computation time. On the other hand, it does not take into consideration all the information available in images. Consequently, the choice of points D and possible errors in the positioning take on greater significance compared with the case where the full cost function is used. In order to limit these effects, the number of points used may be increased. This, however, is made at the expense of a more complicated interaction with the operator.

The function may be minimized by various numeric methods, including, for example, the Brent method for example, which obviates the calculation of the derivatives of the functional and is suitable for computationally efficient execution.

Subsequently, in step 400, the mathematical surface Σ estimated in step 300 may be rasterized; in other words, the three-dimensional mathematical representation which can be described by means of vectors in the system of spherical coordinates (φ,θ) relative to the pole G may be converted into a three-dimensional image which includes a discrete number of voxels.

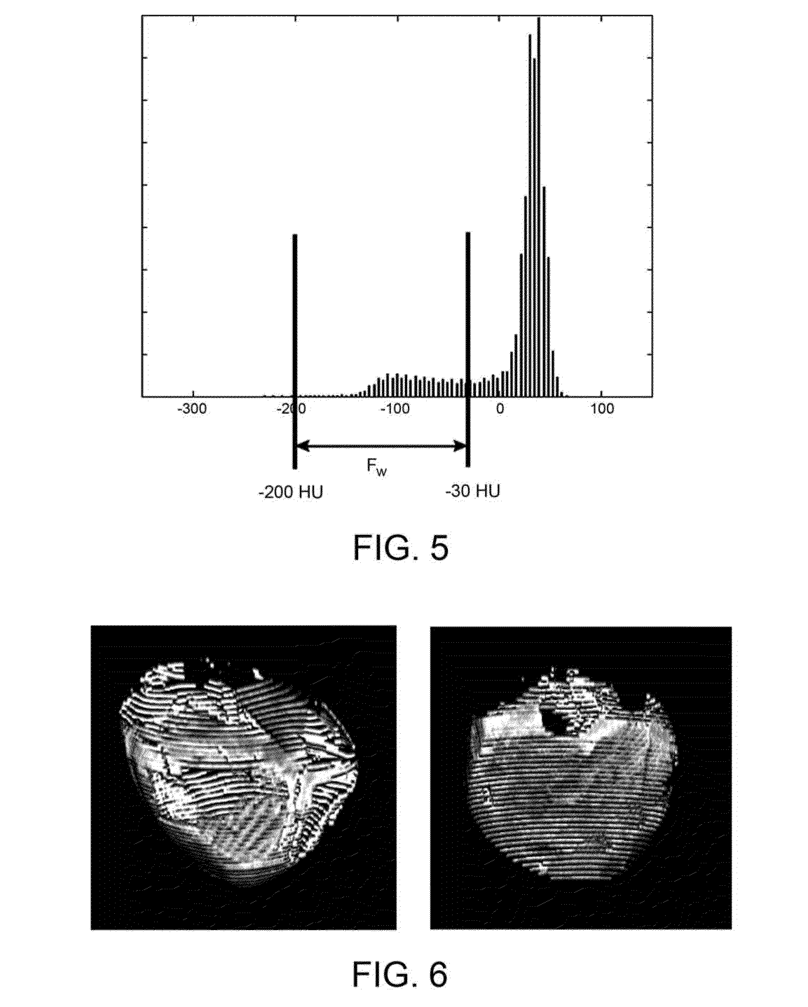

Finally, the calculation of the volume of fat may take place in step 500, and includes counting the voxels which are within the estimated discretized pericardial surface Σ_{d} and have a density within a window [I_{low}, I_{high}] characteristic of fatty tissue, indicated by F_{w} in FIG. 5.

In the case of X-ray tomography, the window F_{w} typically may be in the range from about −200 HU to about −30 HU.

FIG. 6 shows two 3D views (front left and rear right) of the fat deposits identified in this way in the whole epicardial region.

According to certain embodiments, the value of the ratio V/V_{H}, where V_{H} is the total volume of the heart may be provided.

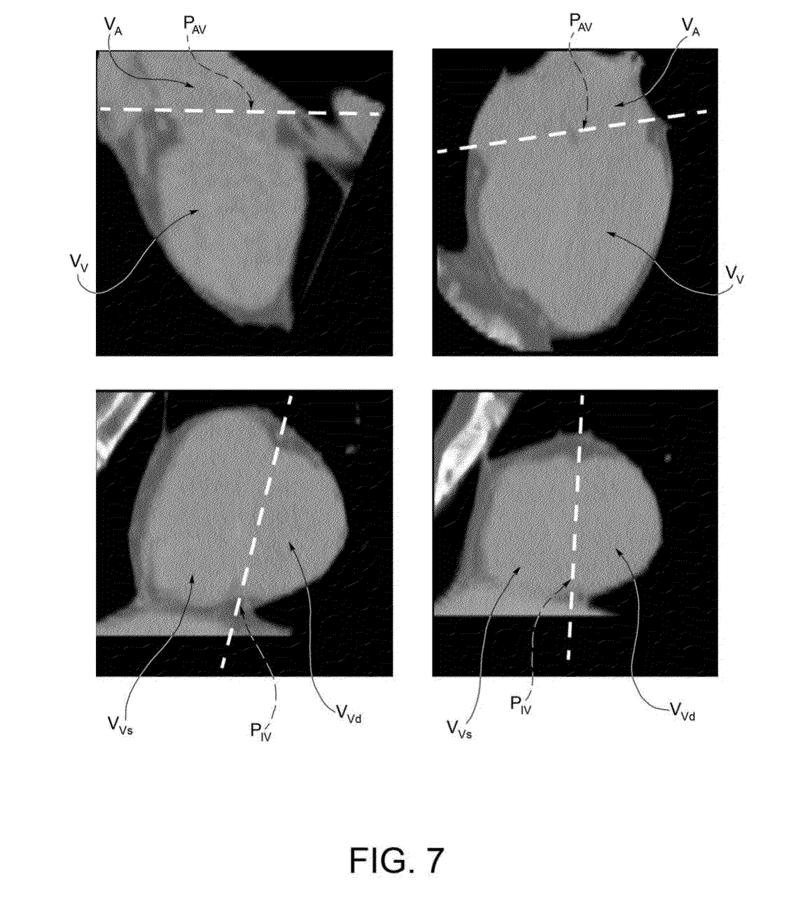

According to certain embodiments, it is also possible to carry out more refined analyses such as the calculation of the regional distribution of fat deposits. For this purpose, the operator may identify the atrioventricular plane P_{AV} (in two long-axis sections as shown in FIG. 7, for example), and the volumes of fat in the ventricular region V_{V} and in the atrial region V_{A} are calculated. Similarly, by identifying the interventricular plane P_{IV} (for example in two short-axis views, also shown in FIG. 7), the volumes V_{Vs} and V_{Vd} of fat in the left and right ventricular regions respectively may be calculated.

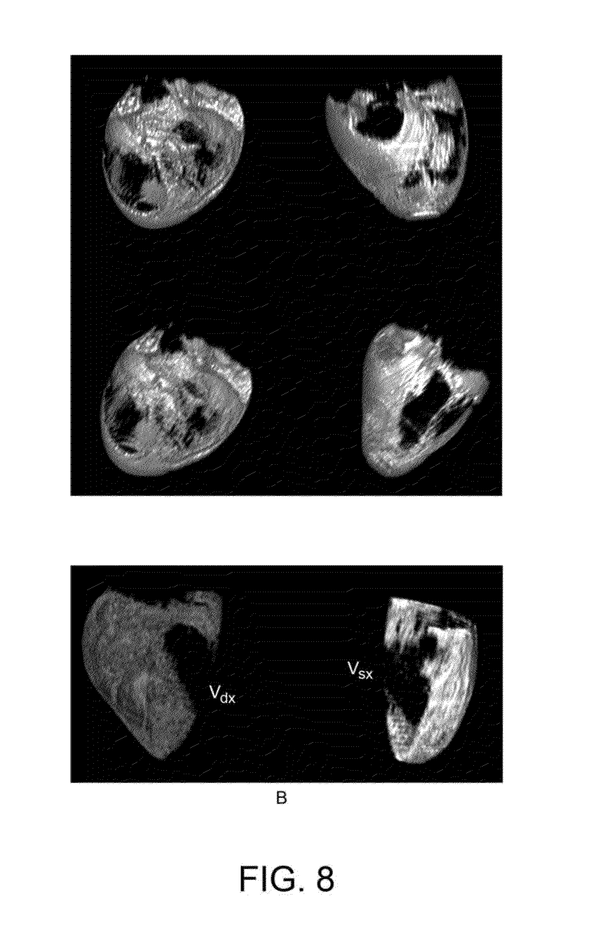

FIG. 8 shows representative 3D views of fat deposits in various cardiac regions. In particular, the upper plate shows the ventricular region from different viewpoints, while the right and left ventricular regions are shown in cross section in the lower plate.

To summarize, methods proposed by the present invention may be used to determine the volume of cardiac fat much more efficiently than previously existing methods, and is characterized by high repeatability, low inter- and intra-operator variability, and substantial rapidity of execution.

Furthermore, methods proposed by the present invention may be applied to volumetric image data obtained either with or without the use of contrast medium.

The methods described herein may be used to improve the process of diagnosing cardiovascular pathologies. In particular, in the case of X-ray tomography, such methods do not require any further exposure to ionizing radiation than the standard procedures. The methods also may be used by non-medical personnel in clinical practice.

Because of the automation of the methods determining the volume of cardiac fat, the invention may be used for studying cardiovascular diseases in large populations.

It should be noted that embodiments of the present invention described herein are purely exemplary and do not limit the present invention. A person skilled in the art may easily apply the teachings disclosed herein to embodiments which do not depart from the principles described above, and which are therefore intended to be included within the scope of the present invention.

This is particularly true of the possibilities for applying the method proposed by the invention to three-dimensional images obtained by different 3D imaging methods.

In particular, no essential modifications are required for magnetic resonance, except in respect of the lower spatial resolution (the method is in itself capable of operating at spatial resolutions other than those of standard CT). It may be necessary to adjust (i) the parameters for the grey window of the fat (in MRI these values depend on the excitation sequence, whereas in CT the variability of the fat window is practically negligible), (ii) the values of the weights α, β, γ, δ of the cost function. It should be noted that some of the constraints expressed in the cost functional can be removed, if necessary, by setting the corresponding coefficients to zero. This may be convenient in cases where the information provided by the grey levels (as in the case of some MRI excitation sequences) and/or by the contours may be confusing. In an extreme case, the method may be used by disregarding the grey level (assuming that β=γ=0) and using only the contours supplied at the input. Naturally, the principle of the invention remaining the same, the forms of embodiment and details of construction may be varied widely with respect to those described and illustrated, which have been given purely by way of non-limiting example, without thereby departing from the scope of protection of the present invention as defined by the present specification and attached claims.

1. A method for determining the volume of epicardial fat of a heart, comprising:
acquiring a plurality of volumetric image data representing the heart;
morphologically characterizing a volume of interest represented by said volumetric image data, which comprises identifying a predetermined number of reference contours of the heart;
estimating the epicardial surface, Σ, according to a three-dimensional model based on a series expansion of vectorial spherical harmonic functions from said reference contours; and
determining the volume of epicardial fat based on voxels which are located inside the estimated epicardial surface, Σ, and which have a grey level within a predetermined range, characteristic of fatty tissue,
wherein estimating the epicardial surface comprises:
expressing the surface as a series of vector spherical harmonic functions in parametric form, as
$$x ( ϕ , θ ) = {\sum }_{n = 0}^{N} {\sum }_{m = 0}^{n} [ {a}_{mn} cos m ϕ + {b}_{mn} sin m ϕ ] {P}_{mn} ( cos ( θ ) )$$
where aₘₙ, bₘₙ are the (vector) coefficients of the series, Pₘₙ(t) are the associated Legendre polynomials of degree n and order m, the number N is the order of the surface, and (φ,θ) are the spherical coordinates with respect to the centroid of the reference contours; and
determining the coefficients of the series expansion by minimizing a cost function selected from the group consisting of:
C(aₘₙ,bₘₙ)=P(aₘₙ,bₘₙ)+λS(aₘₙ,bₘₙ)
wherein
P is a function correlated with approximation error; and
S is a functional correlated with the regularity of the surface and C(aₘₙ,bₘₙ)=αP(aₘₙ,bₘₙ)+βG′(aₘₙ,bₘₙ)+γE(aₘₙ,bₘₙ)+δS(aₘₙ,bₘₙ)
wherein
P is the functional correlated with the approximation error;
G′ is a functional correlated with the presence within the surface of grey levels outside said predetermined range of grey levels of the fatty tissue;
E is a functional correlated with the gradients of grey levels at the interface of the epicardial surface;
S is the functional correlated with the regularity of the surface, and
the coefficients α, β, γ, δ are chosen with the values α, δ=1 and β, γ=10⁻².

2. The method of claim 1, wherein the functional P is calculated as the sum of the distances dᵢ of the reference contours from the epicardial surface, Σ, according to the expression:
$$𝒫 = \frac{1}{K} {\sum }_{i} {d}_{i}^{2} ( {D}_{i} , Σ )$$

3. The method of claim 1, wherein the functional Cis calculated according to the expression:
G′=∫_{Ω}H(I_{high}−I(x,y,z))(I_{high}−I(x,y,x))²dxdydz++∫_{Ω}H(I(x,y,z) . . . I_{low})(I_{low} . . . (x,y,z))²dxdydz
where H is the unitary step function, I(x,y,z) is the grey level function, I_{low}, is the lower bound of the predetermined range of grey levels, and I_{high} is the upper bound of the preset range of grey levels.

4. The method of claim 1, wherein the functional E is calculated according to the expression:
$$ℰ = {\int }_{Σ} \frac{1}{1 + {\vert ∇ I ( x , y , z ) \vert}^{2}} \vert ∇ I ( x , y , z ) \vert d Σ$$
where I(x,y,z) is the grey level function.

5. The method of claim 1, wherein the functional S is calculated according to the expression:
$$𝒮 = {\sum }_{n} {\sum }_{m} {w}_{n m} ( {a}_{m n}^{2} + {b}_{mn}^{2} )$$
where wₘₙ=k exp(n/N).

6. The method of claim 1, wherein said volumetric image data comprise a collection of two-dimensional image data obtained by tomographic or magnetic resonance scanning, representing a plurality of successive plane views of the heart, which, taken in combination, define a grey level function I(x,y,z).

7. The method of claim 1, wherein morphologically characterizing the volume of interest comprises identifying at least three two-dimensional contours of the heart, comprising the profile of the heart in at least a pair of sectional images taken along a long axis, representing four chambers and two chambers respectively, and in at least one sectional image taken along a short axis at the base of the ventricle.

8. The method of claim 7, wherein said contours are identified by applying a method of automatic segmentation of the cardiac volume.

9. The method of claim 7, wherein said contours are identified by the marking of a discrete number of boundary points by an operator and the plotting of an interpolation curve based on the marked points.

10. The method of claim 1, wherein determining the volume of epicardial fat includes counting the voxels inside the estimated surface which has been converted into a three-dimensional image data element comprising a discrete number of voxels.

11. The method of claim 1, comprising determining the regional distribution of the volume of epicardial fat.

12. A system for determining the volume of epicardial fat of a heart, comprising processing means arranged to implement the method of claim 1.

13. A computer program or group of programs executable by a processing system, comprising one or more code modules for implementing a method for determining the volume of epicardial fat of claim 1.

14. A computer program product which stores a computer program or group of programs of claim 14.

15. A method for identifying a subject having a cardiac-related pathology or a subject at risk for having a cardiac-related pathology, comprising applying the method of claim 1 to said subject and determining whether the volume of epicardial fat exceeds normal values.
